(19) United States
(12) Patent Application Publication
(10) Pub. No.: US 2015/0118697 A1
(43) Pub. Date: Apr. 30, 2015

(54) DEVICE AND METHOD FOR THE EXAMINATION OF A SAMPLE FLUID

(71) Applicant: BUERKERT WERKE GMBH, Ingelfingen (DE)

(72) Inventors: Nicole STEIDLE, Karlsruhe (DE); Thomas HAHN, Kunzelsau (DE)

(21) Appl. No.: 14/520,980

(22) Filed: Oct. 22, 2014

(30) Foreign Application Priority Data
Oct. 25, 2013 (DE) 10 2013 111 759.7

(51) Int. Cl. G01N 33/543

(52) U.S. Cl. CPC G01N 33/54386; G01N 33/54306; G01N 2333/4737
USPC 435/7.92; 435/287.2

(57) ABSTRACT

A device for the examination of a sample fluid in a bioanalytical detection method comprises at least one receiving space for the sample fluid and a wall confining the receiving space. The wall is provided with at least one microstructured portion that faces the receiving space and has a multitude of regularly arranged structural elements. The structural elements are shaped in such a way that they form a three-phase border with an aqueous fluid. At the three-phase border at least one biomolecule can be permanently physically adsorbed. The device is in particular a cuvette, a microfluidic chip or a microtiter plate.

### TECHNICAL FIELD

The invention relates to a device and a method for the examination of a sample fluid, and the use of the device.

### BACKGROUND OF THE INVENTION

In so-called immunoassays the presence of a liquid-phase analyte is detected by the bonding of an antigen to an antibody. The analyte's (antigen's) binding partner is usually bound to a solid carrier made of glass or plastic. Glass surfaces are usually conditioned by silanization, and the binding partner is covalenty bound to the silane layer. The immobilization of the binding partner on a plastic surface usually requires a plasma treatment and a subsequent chemical modification of the surface.

Therefore, there remains the need for devices for the performance of bioanalytical methods, in particular microfluidic devices allowing the easy and inexpensive immobilization of test reagents.

### SUMMARY OF THE INVENTION

According to a first aspect of the present invention, a device for the examination of a sample fluid comprises at least one receiving space for the sample fluid and a wall confining this receiving space. The wall comprises at least one microstructured or nanostructured surface portion having a multitude of regularly arranged structural elements, wherein the microstructured or nanostructured surface portion is facing the receiving space. The structural elements are shaped in such a way that they form a three-phase border when being wetted with an aqueous fluid. At least one biomolecule is physically adsorbed in the area of the three-phase border.

In a further aspect, the invention is a method for the examination of a sample fluid using the device such as a bioanalytical detection method, or a biosensor comprising the device.

The invention is based on the finding that the regularly arranged structural elements having dimensions in the nano- or micrometer range can be shaped in such a way that the structural elements, when wetted with an aqueous fluid, do not immerse completely in the fluid. In fact, the fluid is preferably only in contact with the free ends of the structural elements thus forming a three-phase border at the edges on the front end of the structural elements between the fluid, the gaseous phase or air enclosed between the structural elements and the solid material of the structural elements. Thus, at ambient temperature, the fluid does not penetrate into the gas-filled cavity between the structural elements; it is in the so-called Cassie state. The invention also comprises the formation of a non-ideal Cassie state in which the free ends of the structural elements are partially but not completely immersed in the fluid. At the three-phase border an additional force—the line tension—is present, which enables the physical adsorption of biomolecules dissolved in the aqueous solution at the structural elements.

The formation of the three-phase border can be detected by a clearly increased contact angle of the fluid on the nanostructured or microstructured surface portion as compared to an unstructured surface portion of the same material.

Furthermore, it can be shown that biomolecules such as bovine serum albumin (BSA) attach to the structural elements, preferably in the contact area with the aqueous fluid, and are physically adsorbed. Attachment takes place in the area of the three-phase border. It can be determined by means of fluorescent BSA that the biomolecule binds almost exclusively to the structural elements rather than to unstructured portions of the wall. BSA adsorption remains stable also after rinsing with an aqueous solution. Thus, the adsorption is long-term stable even without a covalent chemical bond between the biomolecule and the surface of the structural element. Therefore, the formation of the structural elements according to the present invention facilitates a physical immobilization of biomolecules, without any conditioning of the surface.

However, there remains the possibility, although less preferred, to additionally modify the surface of the structural elements chemically to further stabilize the adsorption of sensitive biomolecules.

According to an embodiment of the invention the structural elements are substantially columnar having a diameter of 0.1 μm to 100 μm and an aspect ratio of 0.1 to 20. The aspect ratio is the ratio of the structural height to the smallest lateral dimension of the structural element.

The substantially columnar structural elements may have a round, preferably a circular or oval cross-section. The cross-section may also be polygonal, for example triangular, quadrangular, hexagonal or octagonal.

According to the present invention, a substantially column-shaped structure also comprises structural elements shaped like a cone or a truncated pyramid.

The distance or lateral spacing between the structural elements is preferred to be at most 4 times, more preferred to be at most 3 times and particularly preferred to be at most 1.5 to 2.5 times its diameter.

Generally, the larger the aspect ratio, the bigger may be the distance between the structural elements. Hydrophobic materials also tolerate a larger distance.

Dimensions of the structural elements in the above mentioned ranges facilitate the formation of a three-phase border with an aqueous fluid applied onto the microstructured or nanostructured surface portion.

Thus, another aspect of the invention is a device for the examination of a sample fluid comprising a receiving space or cavity for the sample fluid and a wall confining the cavity, wherein the wall comprises at least one microstructured or nanostructured section that is facing the fluid and has a multitude of regularly arranged structural elements, with the structural elements being column-shaped having a diameter of 0.1 μm to 100 μm and an aspect ratio of 0.1 to 20, and wherein the distance between adjacent individual structural elements is at most 4 times its diameter.

At their free end facing the cavity, the structural elements have a front surface that has a sharp edge at the junction with the lateral face of the structural elements. Round edges, chamfers and convex areas can cause the fluid to drain off into the space between the structural elements and prevent the formation of the three-phase border. In this case, the microstructured or nanostructured section would be completely wetted.

Preferably, the front surface is substantially planar or concave. The angle between the front surface and the lateral face is preferred to be at most 120°, more preferred to be at most 90°.

According to a particularly preferred embodiment the structural elements have a concave front surface. The concave front surface reduces the contact areas between the fluid and the structural element and additionally facilitates the formation of a three-phase border with the aqueous fluid. Therefore, the concave front surface results in an almost ideal Cassie state. In this state, particularly high line tensions occur, and an effective physical immobilization of the biomolecules at the edges on the front surface of the structural elements is achieved.

The smallest lateral diameter of the structural elements preferably is in the range of from 0.1 μm to 100 μm.

If the diameter of the structural elements is 10 μm, the aspect ratio is at least 1.5. Preferably, the aspect ratio is in the range from 1.5 to 10. Structural elements having a diameter in the nanometer range can be formed with a smaller aspect ratio.

Preferably, the distance between the structural elements is not greater than 3 times and more preferably not greater than 1.5 to 2.5 times its diameter.

According to a preferred embodiment the receiving space is preferably a reaction chamber or a microchannel.

The wall comprising the microstructured or nanostructured section can be made of plastic, glass, metal or ceramics, or of composite materials thereof, for example metallic glasses. There are various methods known for the microstructuring of these materials, for example hot stamping, micro injection molding, powder injection molding or microetching.

Preferably, the wall is made of a thermoplastic material. Examples of thermoplastic materials are polymethylmethacrylate (PMMA), polycarbonate (PC), polysulfone (PSU), polystyrene (PS) or cycloolefin copolymers (COC). The thermoplastic materials are particularly suitable for use in mass production.

It is especially preferred that the wall is at least partially made of an optically transparent plastic. This makes it possible to feed light into the device, for example via optical mirrors, and to photometrically analyze and evaluate a sample introduced in the device.

Preferably, at least one biomolecule is physically immobilized at the structural elements in the microstructured or nanostructured section. The device can then be provided as a prefabricated test kit. Physical immobilization is long-term stable under the conditions of a bioanalytical detection method, i.e. the biomolecule is not detached by treatment with either an aqueous buffer solution, a sample solution or the solution of a detection reagent.

According to a particularly preferred embodiment the wall confining the receiving space is completely provided with the structural elements, thus facilitating a targeted control of the amount of biomolecules immobilized at the structural elements.

The biomolecule physically immobilized at the structural elements is preferably a binding partner that reacts with an analyte in the sample fluid. Examples of such binding partners are streptavidin, avidin, biotin, antibodies, biotinylated antibodies and complexes thereof.

The sites of the structural elements not occupied by the reactive biomolecule can be blocked by another agent that does not react with the analyte. For example, BSA or a water-soluble polymer such as polyethylene glycol or polyvinyl pyrrolidone may serve as a non-reactive agent that is also physically immobilized.

The device for the examination of a sample fluid is preferably a microfluidic chip, a cuvette or a microtiter plate.

A process for the manufacture of the device according to the present invention comprises the steps of initially structuring a plastic film by means of hot stamping, with the structural elements being formed either on the entire film or in a predefined portion of the film surface.

Hot stamping is performed by placing the plastic film into a mold and heating it, with pressure exerted, to a temperature above the glass transition point of the plastic, thus printing the structures preset by the mold into the soft plastic film.

In a next step the plastic film is brought into a predetermined three-dimensional shape by means of thermoforming. By this method, in particular microchannels can be formed in the film.

Thermoforming can be achieved by placing the hot-stamped film into a mold and heating it to a temperature below the glass transition point. The film is formed by pressing nitrogen into the cavity of the mold, thus avoiding a destruction of the structural elements previously incorporated in the film by means of hot stamping.

In a last step the formed film is linked with a carrier plate having all the necessary fluidic connections. Linking of the film with the carrier plate can be achieved, for example, by laser welding but also by other connection technologies such as solvent bonding, thermal bonding or adhesive bonding.

The film and the carrier plate are preferably made of the same material. However, various materials may be used.

Alternatively, the device can also be manufactured using other known methods such as micro injection molding or injection-compression molding.

The device according to the present invention is particularly suitable for the performance of bioanalytical test methods and/or for use as a biosensor.

Preferably, the test method is an immunoassay, and more preferably an enzymatic immunoadsorption method such as ELISA (enzyme-linked immuno sorbent assay). In this method, an enzyme is bound to an analyte, in particular to a complex consisting of an analyte and an antibody. In a detection reaction, a substrate is converted into a detection substrate, in particular a fluorescent substance with the aid of the enzyme. By measuring the detection substrate a quantitative determination of the analyte in the sample fluid is possible.

The analyte binds to a specific biomolecule as a binding partner which is adsorbed at a solid phase. The binding partner may be a complex made of a binding protein such as streptavidin and a binding antibody such as a biotinylated antibody. According to the invention, the microstructured or nanostructured surface portion of the device described above acts as the solid phase. During the performance of the test method the biomolecule remains physically bound to the structural elements. In particular, the biomolecule does not pass into solution.

### DESCRIPTION OF THE DRAWINGS

Further features and advantages of the present invention can be inferred from the following description of preferred embodiments and the drawings which are provided by way of example, only. In the drawings:

FIG. 1 shows a schematic diagram of the device according to the present invention;

FIG. 2 shoes a schematic diagram of a structured portion of the device of FIG. 1;

FIG. 3 shows a schematic diagram of another device according to the present invention;

FIG. 4 shows a schematic diagram of a structural element with immobilized biomolecules.

### DETAILED DESCRIPTION OF PREFERRED EMBODIMENTS

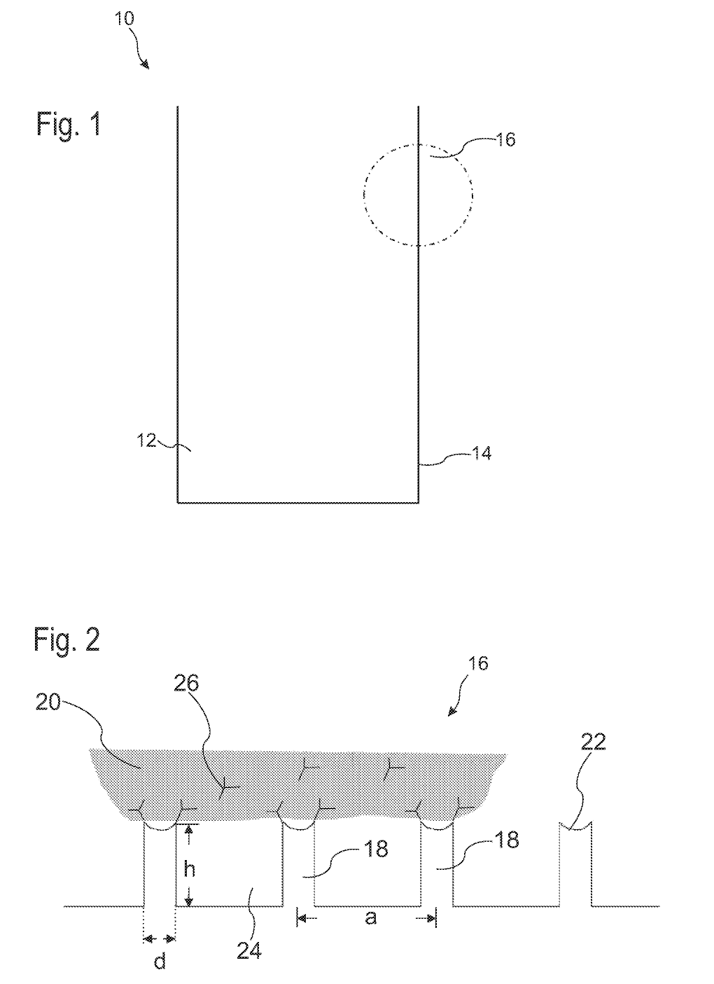

FIG. 1 shows a device for the examination of a sample fluid 10 which, for example, can be a cuvette or the well of a microtiter plate. The device 10 comprises a receiving space 12 for a sample fluid that is confined by the wall 14. The wall includes a microstructured or nanostructured section or portion 16 that, at its surface facing the receiving space 12, is provided with a multitude of regularly arranged structural elements 18.

The nano- or microstructured portion 16 is shown in detail in FIG. 2. The structural elements 18 in section or portion 16 are shaped in such a way that they can form a three-phase border with an aqueous fluid 20 introduced into the receiving space and covering the structural elements. This means that the structural elements 18 do not or only partially immerse in the aqueous fluid 20, and the aqueous fluid 20 does not completely penetrate into the cavity 24 between the structural elements. The structured portion 16 is thus considerably more hydrophobic than non-structured portions of the wall 14, and the fluid 20 shows a clearly increased contact angle in the structured portion 16.

A biomolecule 26 present in the aqueous fluid, due to the line tension prevailing at the three-phase border between the structural element 18, the fluid 20 and the gas-filled cavity 24, can attach to the structural element 18 and is preferably physically adsorbed in the area of the front surface 22 at the free end of the structural elements, without previous chemical modification of the surface.

Physical adsorption of the biomolecule 26 at the structural element 18 is long-term stable. Thus, the biomolecule 26 is physically immobilized at the structural element 18.

As can be further seen in FIG. 2, the structural elements 18 in portion 16 are substantially column-shaped. Preferably, the structural elements 18 have a diameter of 0.1 μm to 100 μm and an aspect ratio of 0.1 to 20. Here, the aspect ratio is the ratio of the structural height h to the smallest lateral diameter d of the structural elements 18.

The diameter of the individual structural elements 18 is preferably 0.1 μm to 50 μm, and the aspect ratio is preferably 0.5 to 10. Preferably, at a diameter of ≧10 μm, the aspect ratio is at least 1.5.

The individual structural elements 18 are regularly spaced apart. The distance between the centers of two neighboring structural elements is at most 4 times, preferably at most 3 times its diameter d. It is particularly preferred that the distance of neighboring structural elements is in the range of 1.5 to 2.5 times its diameter.

The front surface 22 at the free end of the structural elements 18 can be substantially planar. A sharp edge is formed at the junction of the front surface 22 and the lateral surface of the structural elements, without roundings or chamfers, preferably having an angle of at most 120°, more preferably of at most 90° between the front surface and the lateral surface. The sharp-edged shape of the structural elements 18 facilitates the formation of a three-phase border during application of the aqueous fluid 20.

It is particularly preferred that the front surface 22 of the structural elements 18 is concave, as shown in FIG. 2. This embodiment of the structural elements 18 results in an especially high line tension at the three-phase border enabling an effective immobilization of the biomolecules 26 present in the aqueous fluid 20.

Preferably, the device 10 shown in FIGS. 1 and 2 serves as a cuvette or the well of a microtiter plate (not shown here). In particular, the receiving space 12 can be a reaction chamber.

The wall 14 of the device 10 can be made of plastic, glass, metal, ceramics or a composite material thereof. It is particularly preferred that the wall 14 or the entire device 10 is made of a thermoplastic material as this makes the inexpensive mass production of the device possible.

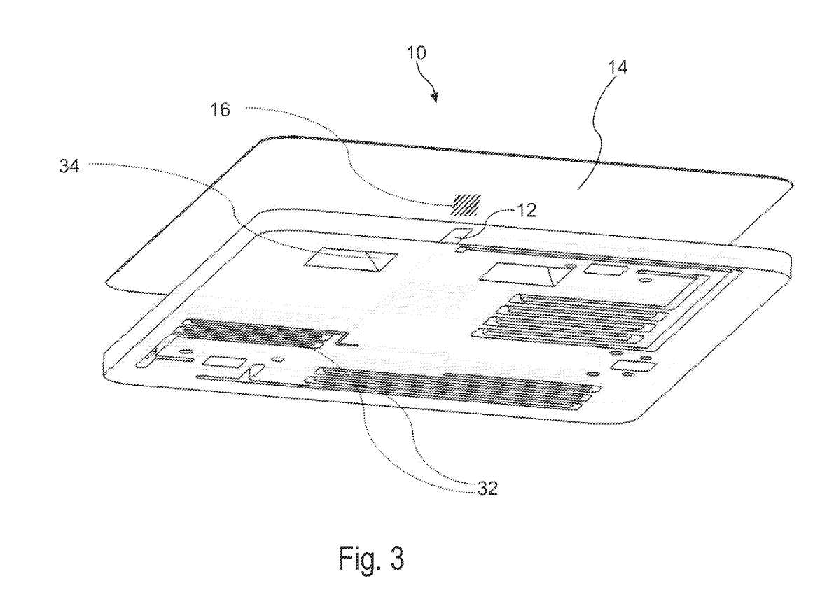

FIG. 3 shows another embodiment of the device 10 according to the present invention in the form of a microfluidic chip. The chip has a wall 14 made of a plastic film that confines a receiving space 12, for example a microchannel or a reaction chamber. A structured portion 16 provided with the structural elements 18 shown and described in FIG. 2 is formed on the plastic film of wall 14.

The plastic film is bonded to a carrier plate 30 having fluidic connections 32 or areas for the retention of fluids. In addition, the chip is provided with an optical mirror 34 through which light can be fed into the device. The light is passed through the receiving space 12 confined by the microstructured portion 16 of the wall 14 and serves the photometric evaluation of the analyte, for example based on fluorescence.

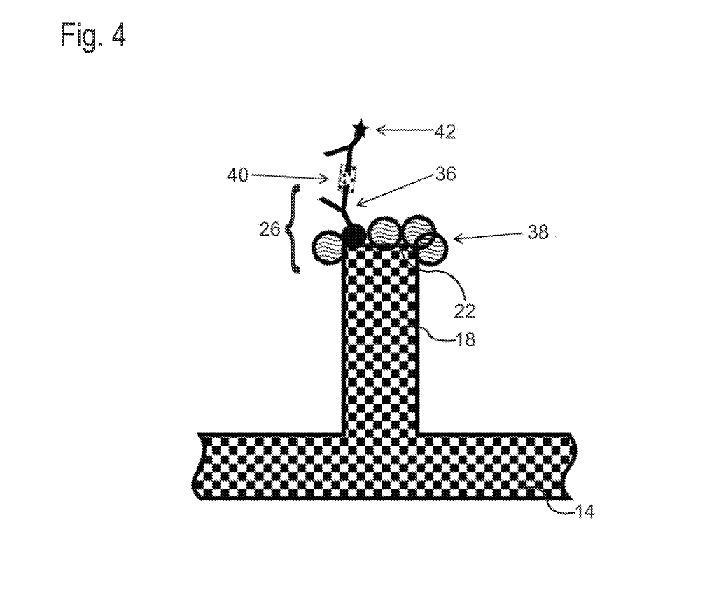

FIG. 4 shows the loading of a structural element 18 with biomolecules 26 during the performance of an ELISA assay.

To prepare the assay, the microstructured or nanostructured portion 16 is brought into contact with an aqueous fluid containing a biotinylated antibody 36, preferably in a buffer solution. The antibody attaches substantially in the area of the front surface 22 of the structural element 18 and is physically immobilized there. Subsequently, washing with a BSA-containing buffer solution causes the BSA 38 to attach to the free sites on the front surface 22 of the structural element 18, which are now blocked for the reception of further biomolecules.

Subsequently, a sample fluid containing the analyte to be examined is passed through the device. The analyte 40, as an antigen, binds to the biotinylated antibody 36.

In the following step the complex comprising the biotinylated antibody 36 and the analyte 40 is reacted with a fluorescence-labeled antibody 42. By using fluorescence the analyte 40 can be detected in the sample. Alternatively, the analyte 40 can also be reacted with an enzyme-linked antibody allowing an enzymatic detection reaction.

The described assay is to be considered only as an example. All known assays requiring a stable immobilization of a binding partner for the analyte at a solid phase can be performed. For example, the biotinylated antibody can also be bound to another binding protein such as streptavidin or avidin that is immobilized on the structural element 18.

The device 10 according to the present invention can be manufactured by injection molding, injection-compression molding, hot pressing and thermoforming or by a combination of these techniques. It is preferred that the wall with the structured portion 16 is manufactured by hot pressing. To this end, a plastic film is placed into a mold containing a stamp with a negative imprint of the portion comprising the structural elements. With pressure exerted, the film is heated in the mold to a temperature above the glass transition point of the plastic and imprinted with the stamp. After cooling the structured plastic film is detached from the stamp.

Following the hot stamping of the structured portion the film can be formed and brought into a desired three-dimensional shape. In so doing, for example additional microchannels and/or reaction chambers can be formed in the film.

Subsequent to the forming step the film is bonded to a carrier plate that may contain fluid reservoirs and fluidic connections. The film and the carrier plate are bonded by means of known methods such as laser welding, thermal bonding, solvent bonding or adhesive bonding.

The immobilization of biomolecules on a plastic film structured according to the present invention was detected with the aid of the assays described below.

Immobilization of BSA

A microchannel with six different successively arranged arrays was formed in a plastic film by means of hot stamping and thermoforming. In each array, columnar structural elements with a structural height of approx. 80 μm were imprinted. The diameter of the structural elements varied from one array to the other and was 10 μm, 15 μm, 20 μm, 25 μm, 30 μm and 35 μm. The distance between the neighboring structural elements was 4 times its diameter.

The structured area of the plastic film was treated with an aqueous solution made of a fluorescence-labeled bovine serum albumin (BSA) at a concentration of 1.25 mg/ml and a buffer solution (phosphate in saline solution, PBS). The mixture of BSA and PBS buffer solution was used at a ratio of 1:19. After 3 hours of incubation the microchannel was rinsed with a pure buffer solution, dried and examined in a fluorescence microscope. A distinct fluorescence could be determined in all arrays.

Immobilization of Streptavidin

A plastic film was microstructured with columnar structural elements by means of hot stamping. The structural elements had a diameter of about 35 μm and an aspect ratio of about 2.5. The distance between the neighboring structural elements was approx. 2 times its diameter. The structured portion of the film was converted into a microchannel and bonded to a carrier plate while forming a microfluidic chip.

Subsequently, a solution of fluorescent streptavidin at a concentration of 10 μg/ml in a PBS buffer solution was passed through the microchannel. Streptavidin immobilization occurs specifically only in that portion of the microchannel which is provided with the columnar structural elements. Permanent adsorption of streptavidin can be detected by fluorescence microscopy.

Blocking with BSA and DNA Detection

A structured surface of a microchannel treated with streptavidin as described before was dried and incubated for 2 hours with an aqueous BSA solution at a concentration of 0.0625 μg/ml in a PBS buffer solution. The BSA occupies the free positions at the columnar structural elements and prevents the attachment of further biomolecules at the columns, thus conditioning the surface of the microchannel in such way that the device can be used in any detection method.

To perform an assay for single-stranded DNA a single-stranded DNA consisting of 22 base pairs with biotin at its 5′ terminal and a red light emitting fluorescent dye at its 3′ terminal is passed through the conditioned chip. The biotin-linked DNA strand binds to the streptavidin immobilized on the surface of the structured portion. In a negative control using a surface only blocked by BSA no attachment of the DNA strand could be detected in the fluorescence microscope.

If the sequence of the DNA strand is complementary to a genetic defect, the presence of a single-stranded DNA indicating one of these genetic defects can be detected in an analyte, for example by means of intercalating fluorescent dyes that are incorporated only in double-stranded DNA.

ELISA Assay

An ELISA assay was used to detect CRP (C-reactive protein) indicating an inflammation in blood samples. In a first step streptavidin was immobilized on the surface of a plastic film structured according to the present invention. After that the free sites on the structured surface were blocked by BSA and a biotinylated antibody was bound to streptavidin.

Subsequently, a CRP-containing analyte at various concentrations was placed in the microfluidic chip conditioned this way. The CRP was bound to the antibody and could be detected by means of another fluorescence-labeled antibody. The various CRP concentrations could be determined by differences in fluorescence intensity.

Desorption Test

As the biomolecules are only physically bound to the nanostructured or microstructured portion, it is possible to reduce the surface tension in the environment of the biomolecules to such an extent that the biomolecules are detached from the structural elements. To detect a desorption, a structured plastic film conditioned with fluorescence-labeled streptavidin was treated with a mixture of isopropanol and water (50% v/v). Fluorescence microscopy showed that streptavidin was almost completely desorbed.

As an alternative to the mixture of isopropanol and water a surfactant solution may be used.

The device according to the present invention is especially suitable for use in bioanalytical detection methods, in particular in the form of a cuvette, a microtiter plate or a microfluidic chip.

In particular, the device according to the present invention can be used in a biosensor that allows both the performance of a detection reaction and its qualitative and/or quantitative evaluation.

The bioanalytical detection method is particularly an immunoassay such as for example an ELISA assay.

As the invention allows a permanent immobilization of biomolecules on structured surfaces, in particular plastic surfaces, the large-scale manufacture of prefabricated test kits is possible. However, the structured surface can also be loaded with specific biomolecules directly by the user. Therefore, various detection methods can be performed using the same device.

While this invention has been described in connection with what is presently considered to be the most practical and preferred embodiment, it is to be understood that the invention is not limited to the disclosed embodiment and the drawings, but, on the contrary, it is intended to cover various modifications and variations within the spirit and scope of the appended claims.

1. A device for the examination of a sample fluid comprising
at least one receiving space for the sample fluid;
a wall confining the receiving space,
at least one microstructured or nanostructured surface section provided in the wall and having a multitude of regularly arranged structural elements, wherein said section faces the receiving space, and wherein the structural elements are shaped in such a way that they form a three-phase border when wetted with an aqueous fluid; and
at least one biomolecule physically adsorbed on the structural elements in the area of the three-phase border.

2. The device according to claim 1 and wherein the structural elements are substantially columnar having a diameter in range of from 0.1 μm to 100 μm and an aspect ratio in a range of from 0.1 to 20 and wherein the structural elements are regularly spaced apart, with the distance between neighboring structural elements being at most 4 times its diameter.

3. The device according to claim 2 wherein the front surface is concave wherein the structural elements comprise a lateral surface and a front surface, and a sharp edge formed at the junction of the lateral surface and the front surface.

4. The device according to claim 1 wherein the structural elements comprise a lateral surface and a front surface, and a sharp edge formed at the junction of the lateral surface and the front surface.

5. A device for the examination of a sample fluid comprising
at least one receiving space for the sample fluid;
a wall confining the receiving space,
at least one microstructured or nanostructured surface section provided in the wall and having a multitude of regularly arranged structural elements, wherein said section faces the receiving space;
wherein each of the structural elements having a substantially columnar shape, a diameter in the range of from 0.1 μm to 100 μm and an aspect ratio in the range of from 0.1 to 20, wherein the structural elements are spaced apart from each other at a distance between neighboring structural elements of at most 4 times its diameter, and wherein the structural elements each having a lateral surface and a front surface, and a sharp edge formed at the junction of the lateral surface and the front surface.

6. The device according to claim 5 wherein a biomolecule is physically adsorbed on said structural elements at least in an area adjacent the sharp edge.

7. The device according to claim 5 wherein the distance between the neighboring structural elements is 1.5 to 2.5 times its diameter.

8. (canceled)

9. The device according to claim 5 wherein the diameter is in the range of from 10 to 100 μm and the aspect ratio is in the range of from 1.5 to 10.

10. The device according to claim 5 wherein the structural elements have a substantially planar front surface.

11. The device according to claim 5 wherein the structural elements have a concave front surface.

12. The device according to claim 5 wherein the structural elements form a three-phase border when wetted with an aqueous fluid.

13. The device according to claim 5 wherein the receiving space is a reaction chamber or a microchannel.

14. The device according to claim 5 wherein the wall is made of a material selected from the group of plastic, glass, metal, ceramics and a composite material thereof.

15. (canceled)

16. (canceled)

17. The device according to claim 5 wherein the wall is completely provided with the structural elements.

18. The device according to claim 6 wherein the biomolecule is selected from at least one of streptavidin, biotin, avidin, BSA, antibodies, biotinylated antibodies, fluorescence-labeled antibodies, proteins and complexes thereof.

19. The device according to claim 5 wherein the device is selected from the group of a microfluidic chip, a cuvette, a microtiter plate, and a biosensor.

20. (canceled)

21. A method for the detection of an analyte in a sample fluid, in which a device according to claim 5 is incubated with a binding partner for the analyte and the binding partner is physically immobilized on the nanostructured or microstructured surface portion.

22. The method according to claim 21 characterized in that the binding partner is selected from the group consisting of streptavidin, biotin, avidin, antibodies, biotinylated antibodies, fluorescence-labeled antibodies and complexes thereof.

23. The method according to claim 21 further comprising the step of treating the nanostructured or microstructured section comprising the physically immobilized binding partner with an aqueous solution containing an agent that does not react with the analyte in order to attach and immobilize the agent on the nanostructured or microstructured section.

24. The method according to claim 23 characterized in that the agent is selected from water-soluble proteins such as BSA and water-soluble polymers such as polyethylene glycol and polyvinyl pyrrolidone.

25. The method of claim 21 characterized in that the method is an immunoassay, in particular an ELISA assay.
